(19) United States
(12) Patent Application Publication
(10) Pub. No.: US 2004/0250329 A1
(43) Pub. Date: Dec. 16, 2004

(54) Hand protector

(76) Inventors: Russell L. Rickert, Upper Darby, PA (US); Leonard R. Rickert, Churchville, PA (US)

Correspondence Address: Gregory J. Gore, Suite 316, 70 W. Oakland Ave., Doylestown, PA, 18901, US

(21) Appl. No.: 10/459,499

(22) Filed: Jun. 12, 2003

(51) Int. Cl. A61F 9/00

(52) U.S. Cl. 2/16; 2/20

(57) ABSTRACT

A hand protector comprises two basic elements, one element attached to the user's body and a second element being a relatively rigid hand guard ergonomically shaped to fit across the palm and fingers of the user's hand. The guard preferably includes a valley region aligned between a palm-covering portion of the guard and an upturned portion at its end so that the grasped elements in hand are maintained in proper position on the guard. The body-securing element is preferably attached to the top of the hand guard at a point adjacent to, or slightly below, the user's wrist. In one embodiment, the body-securing element is a wristband that also contains a snap for releasable attachment to the hand guard element.

### FIELD OF THE INVENTION

The present invention relates to an advertising system which revolves around a simple hand guard which protects a person's hand from discomfort when carrying heavy objects, for example plastic bags which may be filled with groceries or heavy objects such as gathered at trade shows, or paint cans or buckets which have handles with minimal surface areas.

### BACKGROUND OF THE INVENTION

There are known gloves and other types of apparatus for enhancing a person's grip which would also make the handling of grasped objects more comfortable. Such a device is shown, for example, in U.S. Pat. No. 5,634,213 issued to Grover, however devices such as this are clumsy and complicated and furthermore are completely inappropriate for everyday situations involving the carrying of heavy household items in plastic bags and the like. The everyday need is not necessarily for enhancement of the grip but merely for a device which more evenly distributes the forces across the palm of a person's hand. There is therefore a need for an inexpensive, lightweight and convenient hand protector that can make the handling of heavy objects with very narrow handles, such as grocery bags, paint cans, and buckets, more comfortable. By integrating this useful hand guard into an advertising system, this assembly can become very cost effective and available to the everyday user at a reasonable cost.

### SUMMARY OF THE INVENTION

In order to meet the need in the art, the present invention has been devised. It comprises, in its basic form, a minimum of two elements: a flexible securing element attachment to the user's body and a relatively rigid hand guard, ergonomically shaped to fit across the palm and fingers of the hand. The guard preferably includes a valley region aligned between the palm guard and an upturned portion at its end so that the grasped elements are maintained in proper position on the guard. The body-securing element is preferably attached to the top of the hand guard at a point adjacent to, or slightly below, the person's wrist. In its most basic form the body-securing element is connected directly to the top of the hand guard element with a single connecting snap assembly. It is the intention of the invention that in one embodiment, the initial body-securing element (the wrist band) would be a non-reusable, “one day” adjustable plastic wrist strap, incorporating a self-locking device so that the wrist band cannot be removed without destroying itself This wrist band would also contain a compatible snap for attachment to the hand guard element. Such “one day” adjustable plastic wrist straps, with locking devices, that would be used as the initial body securing element are of the type that are commonly used to restrict access at trade shows, air shows and similar events and are typically destroyed by physical removal at the end of the day. To replace these temporary, non-reusable wrist straps, permanent re-useable flexible wrist bands would be available and sold to the users.

In an alternate embodiment, the hand guard may be detachably secured to a resilient and reusable wristband using a ring and fob assembly with a snap compatible with the hand guard. This embodiment would allow for the replacement of the “one day” body securing element wristband with a fully reusable resilient wristband. The use of this reusable body securing element would also provide a method of attaching keys or other useful items such as store supplied bar coded “bonus tags” or various decorative items. This ring and fob arrangement also permits the hand guard to be alternately removed and re-attached to the body-securing element for convenience.

A further embodiment of the body-securing element could be the use of the ring and fob assembly with a compatible hook arrangement that would allow the hand guard to be attached to a person's body using a belt, a belt loop, a shoulder strap, or the like. In all embodiments of the invention, the hand guard element can easily be removed from, and re-attached to, the body-securing element through the use of a set of simple compatible metal or plastic snaps that are used as the primary means of attachment.

The present invention therefore provides the advantage of a convenient hand guard which can be easily and effectively used. Other objects and advantages will be apparent to those of skill in the art from the following drawings and description of the preferred embodiment.

### BRIEF DESCRIPTION OF THE DRAWINGS

FIG. 1 is a top left front isometric view of the present hand guard invention showing the hand guard affixed to a non-reusable, self locking, plastic wrist strap as the body securing element using a compatible snap assembly (this view shows the wrist strap in isolation with the hand strap extended and unlocked).

FIG. 2 is a left side sectional view taken from FIG. 1 depicting the hand guard directly connected to the non-reusable, self locking, plastic wrist strap as the body securing element using a compatible snap assembly.

FIG. 3 is a front left isometric view of the hand guard attached to the non-reusable, self locking, plastic wrist strap, in the locked position, and depicted with the user's hand in phantom.

FIG. 4 is a front left isometric view of the self locking, plastic wrist strap, in the locked position, shown attached to the user's hand in phantom with the hand guard removed.

FIG. 5 is a top front left isometric view of an alternate embodiment of the invention depicting the removal and destruction of the non-reusable, self locking, plastic wrist strap and the replacement with a fully reusable resilient wristband using the attaching ring and fob assembly.

FIG. 6 is a top front left isometric view of an alternate embodiment of the invention depicting the removal and destruction of the non-reusable, self locking, plastic wrist strap and its replacement with a ring and fob assembly.

### DESCRIPTION OF THE PREFERRED EMBODIMENT

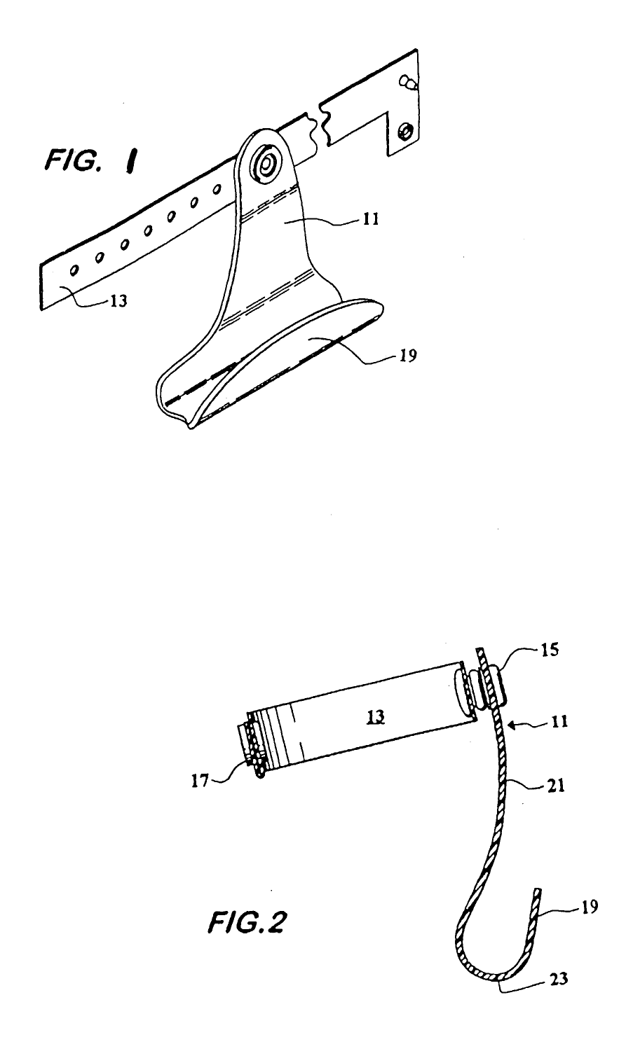

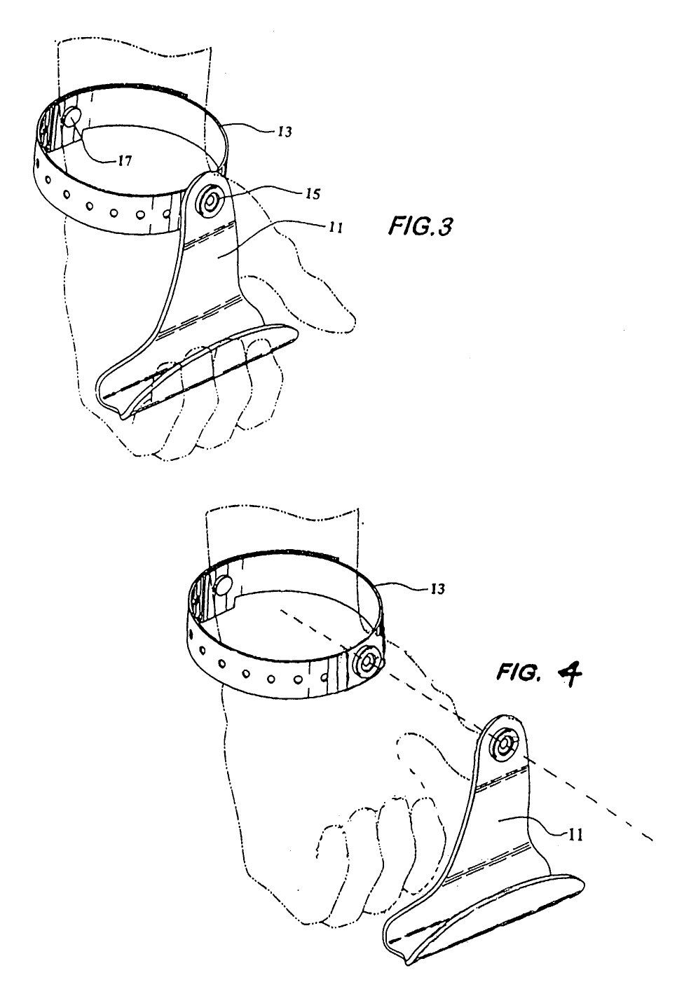

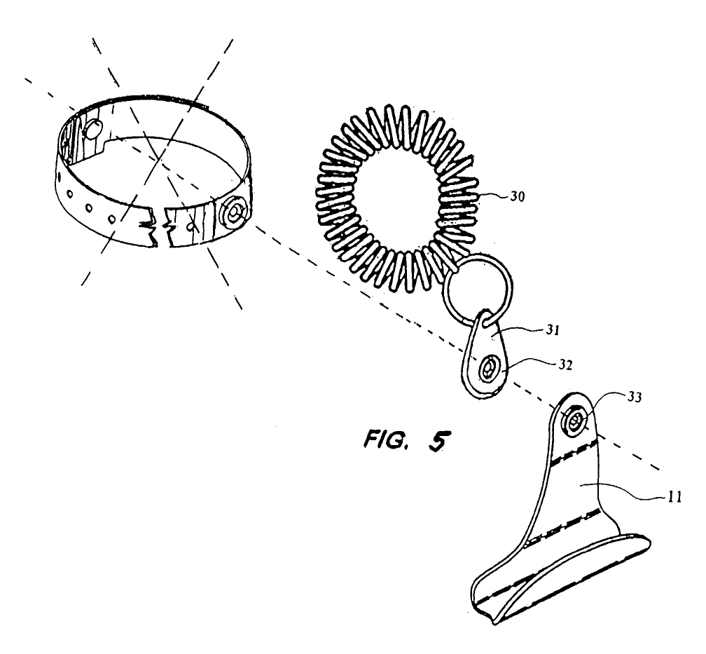

Referring now to FIG. 3, the present invention is shown in position with the hand of the user shown in phantom. The basic elements of the invention are hand guard 11 and a “one day” non-reusable, self locking, plastic wrist strap as the body-securing element 13. Preferably these elements are rotatably and/or releasably joined as for example by a snap means 15. In this embodiment, the wrist strap 13 includes adjustment means 17 so that its length can be altered to fit the size of the user's wrist. However, once the wrist strap is in place and snapped shut, it becomes unusable if removed even though the hand guard 11 can be removed and replaced using the compatible snaps 15 as shown in FIG. 4. Referring now to FIG. 1, the embodiment shown in FIG. 3 is displayed in isolation with “one day” non-reusable, self locking, plastic wrist strap 13 extended and unlocked. The hand guard 11 further includes an upturned lip 19 at its end. This structure is more clearly seen in FIG. 2 which shows greater detail of the hand guard 11 that includes releasable snap means 15 at the top, a convex central portion 21, a containment valley 23, and upturned lip 19 at its end. This is the same embodiment shown in FIG. 3 which includes a “one day” non-reusable, self locking, plastic wrist strap 13 which includes initial length adjustment means 17 and a locking device.

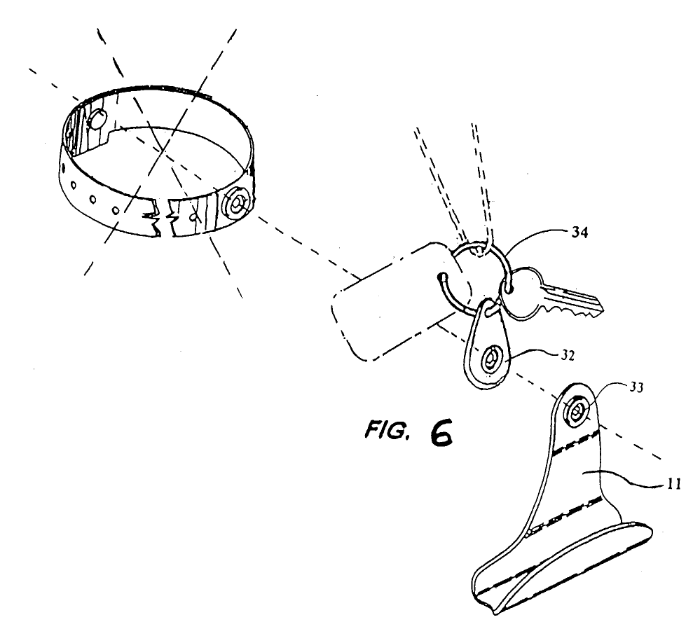

Referring now to FIGS. 4, 5, and 6 alternate embodiments of the invention are shown. The releasable attachment of the hand guard to the body securement means provides the advantages of using existing decorative wrist bands, key chains, and the like rather than a separate dedicated wristband for the hand guard. As shown in FIG. 5, a commonly-used coil wristband 30 and accessory ring includes a fob 31 that has snap attachment means 32 which cooperates with snap attachment means 33 on hand guard 11. Referring now to FIG. 6, a similar arrangement is shown with regard to alternate methods of securing the hand guard 11 to various embodiments of body securing devices using the key ring 34 and fob 32 assembly. While FIGS. 5 and 6 depict alternate embodiments, it will be understood that there are many other variations and adaptations of the detachable hand guard of the invention which may be employed to achieve the advantages and objectives of the invention. In all cases, the wrist guard is preferably composed of a rigid material such as a moldable plastic which is relatively stiff and substantially rigid so that it evenly transfers the forces of the gripped article comfortably across a broad surface of the user's hand. The face of the hand guard and the rear face of the fob may also provide an outward-facing surface to carry advertising, a logo, indicia, or other message. The various modifications and adaptations to the invention that can easily be made are included within the scope of the invention and the preferred embodiments are not meant as limitations. The scope of the invention is to be determined only by the following claims and their legal equivalents.

What is claimed is:

1. A hand protection device, comprising:
a hand guard ergonomically shaped to fit across the palm and fingers of the human hand when in the position of grasping, said guard further including a convex central portion, a containment valley, and an upward-turned lip at its end;
a removable body-securing means which encircles a body part;
attachment means securing said hand guard to said body-securing element.

2. The hand protector of claim 1 wherein said attachment means releasably affixes said hand guard to said body-securing means.

3. The hand protector of claim 2 wherein said body-securing means is a wrist strap or other suitable retaining device.

4. The hand protector of claim 3 wherein said attachment means rotatably joins said hand guard and said body-securing element.

5. The hand protector of claim 4 wherein said attachment means provides snap attachment between the hand guard and the body-securing means.

6. The hand protector of claim 5 wherein said body-securing means includes an adjustable length strap with appropriate connectors.

7. The hand protector of claim 6 wherein said face of the hand guard includes an advertising message.

8. The hand protector of claim 7 wherein said rear face of the connecting fob includes an advertising message.

9. A hand protection device, comprising:
a hand guard ergonomically shaped to fit across the palm and fingers of the human hand when in the position of grasping, said guard further including a convex central portion, a containment valley, and an upward-turned lip at its end;
a removable body-securing means which is attached to a clothing part from the group consisting of a belt, a belt loop, or a strap;
attachment means securing said hand guard to said body-securing element.
